(19) United States
(12) Patent Application Publication
(10) Pub. No.: US 2023/0301583 A1
(43) Pub. Date: Sep. 28, 2023

(54) SEVERITY RATING SYSTEM FOR PORT-WINE STAINS AND METHOD THEREFOR

(71) Applicant: SHANGHAI FUDAN-ZHANGJIANG BIO-PHARMACEUTICAL CO., LTD., Shanghai (CN)

(72) Inventors: Wenhui HOU, Shanghai (CN); Jining TAO, Shanghai (CN); Yi ZHAO, Shanghai (CN); Jianping JIANG, Shanghai (CN); Yong SU, Shanghai (CN)

(21) Appl. No.: 18/014,650

(22) PCT Filed: Jul. 6, 2021
(86) PCT No.: PCT/CN2021/104723

§ 371 Date: Jan. 5, 2023

(30) Foreign Application Priority Data
Jul. 9, 2020 (WO) PCT/CN2020/101170

(51) Int. Cl. A61B 5/00; A61B 5/103; G16H 50/30

(52) U.S. Cl. CPC A61B 5/444; A61B 5/0053; A61B 5/1032; G16H 50/30

(57) ABSTRACT

A severity rating system and method for port-wine stains (PWS), the system comprising: an indicator selection and assignment module, an indicator processing module, and a rating module. The indicator selection and assignment module selects rating indicators for divided rating ranges and assigns values to the indicators, the indicator processing module scores the severity of a PWS, and the rating module rates the severity. The severity rating system and method for PWS comprehensively, completely and objectively consider multiple factors that reflect the severity of PWS; in addition, the accuracy and reproducibility of the rating results are high.

### TECHNICAL FIELD

The present disclosure relates to a severity rating system for port-wine stains and a method therefor.

### BACKGROUND

Nevus flammeus, also known as port-wine stains (PWS), commonly known as birthmarks, are congenital malformations caused by dilated capillaries in the superficial dermis, with an incidence of 0.3%-0.5%. PWS often appears at birth or shortly after birth, and mainly occurs on the face, neck and scalp, with the distribution areas of trigeminal nerve, cervical nerve and spinal nerve being the most common. PWS usually appears as bright red patches in the early stage, which deepen in color with age and become purplish red, thicken into plaques and even nodules. The histopathological manifestations are clusters of dilated capillaries in the upper and middle dermis without endothelial cell proliferation. PWS rarely regresses spontaneously, if left untreated, it will generally accompany the patient throughout life, causing serious impact on the patient’s psychology and quality of life.

Treatment methods for port-wine stains include laser, freezing, excision followed by skin grafting, and the newer photodynamic therapy (PDT). In order to accurately evaluate the efficacy of new therapies in clinical trials, the severity of PWS in patients must first be accurately and uniformly rated.

In the field of dermatology, there are several rating systems for the severity of pigmentation, such as the PASI (Psoriasis Area and Severity Index), MASI (Melasma Area and Severity Index) and EASI (Eczema Area and Severity Index) rating systems for psoriasis. All three rating systems are static and quantitative. The so-called static means that the results of the previous evaluation will not be taken into account in a given evaluation, but only the current observed symptoms will be rated. In contrast to static rating, the so-called dynamic efficacy evaluation refers to correlating the patient’s symptoms over time by setting a baseline, it usually evaluates the efficacy with the description of “better than baseline” or “worse than baseline”.

At present, there is no static method for rating the severity of PWS clinically, and dynamic evaluation of efficacy is usually performed directly in clinical trials. For example, three third-party medical experts, unrelated to the current clinical trial, evaluate the efficacy based on pre- and post-treatment photos of the patients under the condition that they are blinded to the trial treatment assignment.

The total effective rate is used as the main efficacy indicator in the evaluation. The criteria for judging the efficacy are as follows: substantially healed: the color of the treatment area substantially disappeared (degree of improvement ≥ 90%); markedly effective: the color of the treatment area markedly disappeared (degree of improvement ≥ 60%, < 90%); improved: the color of the treatment area partially disappeared (degree of improvement ≥ 20%, < 60%); ineffective: the color of the treatment area basically did not change (degree of improvement < 20%). Finally, the calculations are as follows: substantially healed rate = number of the substantially healed/total number of cases × 100%; total markedly effective rate = (number of the substantially healed + number of the markedly effective)/total number of cases × 100%; total effective rate = (number of the substantially healed + number of the markedly effective + number of the improved)/total number of cases × 100%.

This method essentially uses the pre-treatment photos as the baseline, and compares the post-treatment photos with the baseline, so as to describe the degree of healing after treatment relative to that before treatment. Under this method of efficacy evaluation, an expert relies more on experience to give a “difference” in the severity of the patient’s disease before and after treatment, and it is difficult for a person to be objective and consistent in his or her observation of the difference.

In addition, although there is a set of standard operating procedures in clinic to guide the photographing of patients before and after treatment, only a rough judgment can be made based on the above method since the photos cannot accurately show the texture and thickness of the skin of the focus, the color of the focus may change depending on the light and distance, and the display screen resolution or printing quality of the photos may also cause color difference.

In conclusion, for PWS, since there is no static rating system using in vivo observation (i.e., clinicians directly observe patients (rather than photos)), it is difficult to draw accurate conclusions about the efficacy by dynamic evaluation based only on the degree of color fading in the treatment area. This will greatly affect the development of new therapies.

Based on the above clinical needs, it is required to develop a method that can provide a static rating of the severity of port-wine stains. The method should be able to assist physicians in making a comprehensive and objective judgment of the focus and help physicians to accurately measure the effectiveness of the treatment.

### CONTENT OF THE PRESENT INVENTION

In response to the shortcomings that there is no method to statically rate the severity of port-wine stains in the prior art, the present disclosure provides a severity rating system for port-wine stains and a method therefor, which can statically rate the severity of port-wine stains with high accuracy of the rating results, by taking multiple factors (color, area, hypertrophy, number of nodules, location) that reflect the severity of PWS into account in a comprehensive and complete manner.

A first aspect of the present disclosure relates to a severity rating system for port-wine stains, comprising: an indicator selection and assignment module, an indicator processing module, and a rating module, wherein: the indicator selection and assignment module selects a rating indicator for a divided rating range and assigns a value to the indicator, the indicator processing module scores the severity of a port-wine stain focus, and the rating module rates the severity; wherein, the indicator processing module calls data in the indicator selection and assignment module to score the severity of the port-wine stain focus, followed by inputting the scoring result into the rating module, and the rating module outputs a rating result of the severity;

the division of the rating range refers to dividing one or more areas to be examined on skin of a rating subject, and the area to be examined is an area that is geometrically contiguous without overlapping and that contain or may contain a focus of a port-wine stain.

In a preferred embodiment, the rating indicator in the indicator selection and assignment module comprises: a size and a hypertrophy.

The size refers to an area percentage of the focus in the area to be examined.

The hypertrophy refers to a protruding degree of a skin surface of the focus relative to a skin surface of a healthy site in the area to be examined.

In a more preferred embodiment, the rating indicator further comprises a color; the color refers to a degree of color darkening of an epidermal layer of a site of the focus relative to an epidermal layer of the healthy site in the area to be examined.

In a further preferred embodiment, the rating indicator further comprises a number of nodules and/or a location.

The number of nodules refers to number of palpable localized and substantial skin lesions.

The location refers to a site where the focus is located on the body of the rating subject.

In the indicator selection and assignment module, assigning values to the rating indicators refers to assigning values to selected indicators in accordance with an assignment method;

the assignment method specifically comprises:

the size is divided into S intervals in order from small to large according to a percentage of the focus in the area to be examined, wherein S ≥ 2; preferably 2 to 7; more preferably 4 to 6; when a value of the interval Sᵥ = 0, the percentage of the interval contains 0 and there is no clinically significant focus; when Sᵥ is a maximum value, the percentage of the interval contains 100%, that is, a site of the focus fully occupies the area to be examined.

The hypertrophy is divided into H gradients according to a visually identifiable protruding degree of a skin surface of the focus relative to a skin surface of a healthy site of the area to be examined, H ≥ 2; preferably 2 to 4; when a value of the gradient Hᵥ = 0, the protruding degree is 0, when Hᵥ is a maximum value, the protruding degree is a marked protrusion.

In a preferred embodiment, the color is divided into C categories in order from light to dark according to the degree of color darkening of an epidermal layer of the focus, C ≥ 2; preferably 4 to 6; when a value of the category Cᵥ = 0, the color is a healthy color, when Cᵥ is a maximum value, the color is purple-black.

In a more preferred embodiment, the number of nodules is divided into N groups according to the number of palpable localized and substantial skin lesions, N ≥ 2; when a value of the number Nᵥ = 0, there are no nodules, when Nᵥ is a maximum value, the number of nodules is greater than 10.

In a more preferred embodiment, the location is divided into L grades according to the site where the focus is located on the body of the rating subject, L ≥ 2; preferably 2 to 6, more preferably 3 to 4; when a value of the grade Lᵥ = 0, the focus is located below the neck, when Lᵥ is a maximum value, the lesion is located in middle of a face.

The grade of the location depends on the impact of the location on the appearance of the rating subject.

In the indicator selection and assignment module, preferably, when S = 5, Sᵥ ranges from 0 to 4, when Sᵥ = 0, the percentage is 0-20%; when Sᵥ = 1, the percentage is 20-40%; when Sᵥ = 2, the percentage is 40-60%; when Sᵥ = 3, the percentage is 60-80%; when Sᵥ = 4, the percentage is 80-100%.

When H = 4, Hᵥ ranges from 0 to 3, Hᵥ = 1 means a degree of protrusion that is difficult to be visually identified but palpable by palpation; Hᵥ = 2 means a little protrusion that is visually identifiable; Hᵥ = 3 means a marked protrusion that is visually identifiable.

More preferably, when C = 5, Cᵥ ranges from 0 to 4, when Cᵥ = 0, the color is a healthy color; when Cᵥ = 1, the color is pink that is slightly darker than a color of the healthy site; when Cᵥ = 2, the color is red; when Cᵥ = 3, the color is purple; when Cᵥ = 4, the color is purple-black.

Further more preferably, when L = 4, Lᵥ ranges from 1.0 to 1.3, Lᵥ = 1.0 means that the focus is at a site below a neck; Lᵥ = 1.1 means that the focus is located in the neck; Lᵥ = 1.2 means that the focus is located in periphery of the face; Lᵥ = 1.3 means that the focus is located in the middle of the face.

Alternatively, when L = 3, a range of Lᵥ is (0, 0.1, 0.3), Lᵥ = 0 means that the focus is located on a non-face site; Lᵥ = 0.1 means that the focus is located on a chin; Lᵥ = 0.3 means that the focus is located on a forehead or a left cheek or a right cheek.

In the indicator processing module, scoring the severity refers to: performing a quantitative calculation and weighting on each area to be examined according to weight and assigned value of each indicator of the area to be examined to obtain a score of the severity of the rating range.

Preferably, a formula for the quantitative calculation is as follows:
(1) when weights of the selected indicators are size, color and hypertrophy in descending order:
   X₁ = (Cᵥ + Hᵥ) × Sᵥ (formula 1), wherein, the severity is a lowest level when X₁ = 0; the severity is a highest level when X₁ = [(C - 1) + (H - 1)] × (S - 1); or
(2) when the weights of the selected indicators are color, hypertrophy and size in descending order:
   X₂ = 0.7 × Cᵥ + 0.2 × Hᵥ + 0.1 × Sᵥ (formula 2), wherein, the severity is a lowest level when X₂ = 0; the severity is a highest level when X₂ = (0.7 × C + 0.2 × H + 0.1 × S) - 1.

More preferably, when the selected indicator further comprises location:

$${\text{X}}_{1} \text{} ′ = {\text{X}}_{1} \times {\text{L}}_{\text{v}} ; {\text{X}}_{2} \text{} ′ = {\text{X}}_{2} \times {\text{L}}_{\text{v}} .$$

When the selected indicator further comprises the number of nodules, there are two calculation methods:
calculation method 1: value of the number of nodules is included in hypertrophy indicator: formula of the area to be examined is X₃ = Lᵥ × Sᵥ × (Cᵥ + Hᵥ) (formula 3);
calculation method 2: the value of the number of nodules is calculated separately: the formula of the area to be examined is X₃′ = Lᵥ × Sᵥ × (Cᵥ + Hᵥ + Nᵥ).

Results obtained by the two calculation methods are the same.

Severity level = severity level when the number of nodules is not included (Level) + Nᵥ

The severity is rated on a scale of at least three consecutive and independent levels from mild to severe, the lowest level representing that the rating subject does not have PWS or that the focus has been cured in clinical sense; the highest level representing that the focus is the most severe PWS in clinical sense.

In a specific embodiment, when C = 5, S = 5, H = 4, the severity of the focus can be classified into five successive and independent levels from mild to severe;
when X₁ = 0-5, the severity of the site of the focus is the lowest level (i.e., normal in Table 2); when X₁ = 6-11, the severity of the site of the focus is a second lowest level (i.e., mild); when X₁ = 12-17, the severity of the site of the focus is an intermediate level (i.e., moderate); when X₁ = 18-23, the severity of the site of the focus is a second highest level (i.e. severe); when X₁ = 24-28, the severity of the site of the focus is the highest level (i.e., extremely severe); or
when X₂ = 0-0.76, the severity of the site of the focus is the lowest level; when X₂ = 0.77-1.52, the severity of the site of the focus is a second lowest level; when X₂ = 1.53-2.28, the severity of the site of the focus is an intermediate level; when X₂ = 2.29-3.04, the severity of the site of the focus is a second highest level; when X₂ = 3.05-3.80, the severity of the site of the focus is the highest level.

In a preferred embodiment, number of areas to be examined is ≥ 1, preferably from 1 to 10, for example 2, 3, 4, 5 or 6.

A geometry of the area to be examined may be conventional in the art, such as a square, a rectangle or a circle.

In a more preferred embodiment, a rating range is pre-defined prior to the division of the rating ranges.

The area to be examined is comprised in the rating range.

The rating range may be conventional in the art, such as a face or a whole body.

In some preferred embodiments, the rating system also comprises a result printing module, the result printing module prints the rating result output by the rating module.

Preferably, the rating system also comprises an input module, the input module can provide an input image result to the indicator selection and assignment module.

In some preferred embodiments, the system further comprises an auxiliary tool for quantifying and assigning values to each indicator, preferably a colorimetric standard for assisting in quantifying and assigning values to the indicator of the color and a measuring tool for assisting in quantifying and assigning values to the indicator of the size.

The colorimetric standard may be a flat print or a substantially spherical three-dimensional print; preferably a paper color card of a flat print.

At least C color blocks are printed sequentially on the colorimetric standard according to the color from light to dark, the characteristic color of each of the categories is the same as color of at least one color block. The color blocks may be circular or apple-shaped.

The colorimetric standard (taking the color card as an example) is used in a way similar to pH test paper, the color card is placed close to the site of the focus, each color block is compared with the color of the focus one by one to check if their colors are similar, the most similar color block is selected, and the quantitative result of the color indicator is a category represented by the color block.

The measuring tool is a flat print of the same shape as the area to be examined, on which checkerboard-shaped square grids of the same size are printed all over, such as a grid paper.

The measuring tool is preferably a soft transparent or translucent material.

The measuring tool is used by applying the grid paper over or near the area to be examined, counting number of grids in which the site of the focus overlaps, and dividing the number of grids by the total number of grids to obtain the percentage of the site of the focus, that is, quantitative data of the size indicator.

A second aspect of the present disclosure relates to a rating method based on the system in the first aspect, comprising following steps:
P1: defining a rating range comprising one or more areas to be examined on skin of a rating subject, and the area to be examined is an area that is geometrically contiguous without overlapping and that contain or may contain a focus of port-wine stains;
number of the areas to be examined is ≥ 1, preferably from 1 to 10, for example 2, 3, 4, 5 or 6.

A geometry of the area to be examined may be conventional in the art, such as a square, a rectangle or a circle.

The rating range may be conventional in the art, such as a face or a whole body.

P2: performing a numerical quantitative assignment of at least one indicator of the skin of a site of the focus of the port-wine stains defined in P1 by calling an indicator selection and assignment module.

The rating indicator comprises: a size and a hypertrophy; preferably, a color; more preferably, a number of nodules and/or a location.

The size refers to an area percentage of the focus in the area to be examined.

The hypertrophy refers to a protruding degree of a skin surface of the focus relative to a skin surface of a healthy site in the area to be examined.

The color refers to a degree of color darkening of an epidermal layer of a site of the focus relative to an epidermal layer of the healthy site in the area to be examined.

The number of nodules refers to number of palpable localized and substantial skin lesions.

The location refers to a site where the focus is located on the body of the rating subject.

Assigning values to the rating indicators refers to assigning values to selected indicator in accordance with an assignment method;
the assignment method is specifically as follows:
the size is divided into S intervals in order from small to large according to a percentage of the focus in the area to be examined, wherein S ≥ 2; preferably 2 to 7; more preferably 4 to 6; when a value of the interval Sᵥ = 0, the percentage of the interval contains 0 and there is no clinically significant focus; when Sᵥ is a maximum value, the percentage of the interval contains 100%, that is, the site of the focus fully occupies the area to be examined.

The hypertrophy is divided into H gradients according to a visually identifiable protruding degree of the skin surface of the focus relative to the skin surface of the healthy site of the area to be examined, H ≥ 2; preferably 2 to 4; when a value of the gradient Hᵥ = 0, the protruding degree is 0, when Hᵥ is a maximum value, the protruding degree is a marked protrusion.

The color is divided into C categories in order from light to dark according to a degree of color darkening of the epidermal layer of the site of the focus, C ≥ 2; preferably 4 to 6; when a value of the category Cᵥ = 0, the color is a healthy color, when Cᵥ is a maximum value, the color is purple-black.

The number of nodules is divided into N groups according to the number of palpable localized and substantial skin lesions, N ≥ 2; when a value of the number Nᵥ = 0, there are no nodules, when Nᵥ is a maximum value, the number of nodules is greater than 10.

The location is divided into L grades according to the site where the focus is located on the body of the rating subject, L ≥ 2; preferably 3 to 4; when a value of the grade Lᵥ = 0, the focus is located below a neck, when Lᵥ is a maximum value, the focus is located in the middle of the face.

The grade of the location depends on the impact of the location on the appearance of the rating subject.

Preferably, when S = 5, Sᵥ ranges from 0 to 4, when Sᵥ = 0, the percentage is 0-20%; when Sᵥ = 1, the percentage is 20-40%; when Sᵥ = 2, the percentage is 40-60%; when Sᵥ = 3, the percentage is 60-80%; when Sᵥ = 4, the percentage is 80-100%.

Preferably, when H = 4, Hᵥ ranges from 0 to 3, Hᵥ = 1 means a degree of protrusion that is difficult to be visually identified but palpable by palpation; Hᵥ = 2 means a little protrusion that is visually identifiable; Hᵥ = 3 means a marked protrusion that is visually identifiable.

Preferably, when C = 5, Cᵥ ranges from 0 to 4, when Cᵥ = 0, the color is a healthy color; when Cᵥ = 1, the color is pink that is slightly darker than a color of the healthy site; when Cᵥ = 2, the color is red; when Cᵥ = 3, the color is purple; when Cᵥ = 4, the color is purple-black.

Preferably, when L = 4, Lᵥ ranges from 0 to 3, Lᵥ = 0 means that the focus is located at a site below the neck; Lᵥ = 1 means that the focus is located in the neck; Lᵥ = 2 means that the focus is located in periphery of the face; Lᵥ = 3 means that the focus is located in middle of the face.

P3: calculating, by an indicator processing module, numerical rating results of the severity of the port-wine stains in the area to be examined based on results of the quantitative assignment of indicators of the skin in P2, and outputting the numerical rating results through a rating module.

A formula for the quantitative calculation is as follows:

$${\text{X}}_{1} = \left({\text{C}}_{\text{v}} + {\text{H}}_{\text{v}}\right) \times {\text{S}}_{\text{v}} \qquad \text{(formula 1)}$$

or when the indicators color, size and hypertrophy are ranked in descending order of weight as color, size and hypertrophy: X₂ = 0.7 × Cᵥ + 0.2 × Hᵥ + 0.1 × Sᵥ (formula 2).

When the selected indicator futher comprises location: X₁′ = X₁ × Lᵥ; X₂′ = X₂ × L_{v.}

When the selected indicator further comprises the number of nodules, there are two calculation methods:
calculation method 1: value of the number of nodules is included in hypertrophy indicator: formula of the area to be examined is X₃ = Lᵥ × Sᵥ × (Cᵥ + Hᵥ) (formula 3);
calculation method 2: the value of the number of nodules is calculated separately: the formula of the area to be examined is X₃′ = Lᵥ × Sᵥ × (Cᵥ + Hᵥ + Nᵥ).

Results obtained by the two calculation methods are the same.

Severity level = severity level when the number of nodules is not included + Nᵥ

The severity is rated on a scale of at least three consecutive and independent levels from mild to severe, the lowest level representing that the rating subject does not have PWS or that the focus has been cured in clinical sense; the highest level representing that the focus is the most severe PWS in clinical sense.

The numerical rating results of the severity of port-wine stains of the rating range refer to a sum of the numerical rating results of the severity of port-wine stains of each area to be examined according to weights.

A third aspect of the present disclosure relates to a computer-readable storage medium storing a computer program, wherein the computer program implements steps of the method as described in the second aspect when executed by a processor.

The computer-readable storage medium may be conventional in the art, preferably a computer hard disk or a USB disk.

In the present disclosure, quantitative assignments are performed visually. Observation can be carried out either directly in front of the rating subject, or in front of photos of the area to be examined. The visual observation can be supplemented by palpation, instrument measurement and template comparison to improve the accuracy of the quantitative assignment.

The positive progressive effect of the present disclosure is that:

the present disclosure provides a set of static and quantitative severity rating methods for port-wine stains.

Using the rating methods, those skilled in the art can directly and objectively evaluate the severity of a patient’s PWS during an in-person consultation, without the need for a third-party expert to evaluate it through photos, which improves the consistency of the evaluation conclusions of different personnel. The judgment of the condition is not affected even if the patient is not treated by the same physician at all stages of the consultation, which greatly reduces the difficulty of the patient’s consultation, and helps to maintain the consistency of the treatment plan.

For the same physician, the rating method can also minimize the influence of his or her experience on the rating results, thus ensuring that there is less variation in the ratings made by the same physician in both early and late stage during a long-term clinical trial.

The present disclosure takes multiple factors (color, area, hypertrophy, number of nodules and location) that reflect the severity of PWS into account in a comprehensive and complete manner, and the accuracy of the rating results is high.

The operation of scoring the severity of PWS with the help of the rating system and rating method of the present disclosure is convenient and fast, and can be quickly mastered by clinicians through short-term training. The scoring time for each patient can be controlled within 2-3 minutes.

The impact of PWS on the patient’s quality of life is mainly reflected in the appearance, and the assignment method of several indicators with the highest weight of the present disclosure is visual observation, which ensures that the physician’s judgment of the symptoms is consistent with the patient’s own feelings, and avoids the distress caused by the inconsistency between medical judgment and the patient’s quality of life perception.

### BRIEF DESCRIPTION OF THE DRAWINGS

FIG. 1 shows a schematic diagram of the present disclosure.

FIG. 2 shows a schematic diagram of 4 areas to be examined defined in face in Embodiment 4.

FIG. 3 shows a division of categories of port-wine stains according to color indicator in Embodiment 4 (C = 5).

FIG. 4 shows a division of gradients of port-wine stains according to hypertrophy indicator in Embodiment 4 (H = 4).

FIG. 5 shows a comparison of time required to perform quantitative assignments for six raters in week 1 and week 2 in Embodiment 4.

FIG. 6 shows correlation between a quantitative rating result X₃ and a PGA rating result in Embodiment 4.

FIG. 7 shows a schematic diagram of a face-to-face diagnosis photo in Embodiment 4 (treated for port-wine stains).

FIG. 8 shows a schematic diagram of a face-to-face diagnosis photo in Embodiment 4 (not treated for port-wine stains).

FIG. 9 shows a schematic diagram of Embodiment 6;
in the figure, 9 is an electronic device, 91 is a processor, 92 is a memory, 93 is a bus, 94 is an external device, 95 is an I/O interface, and 96 is a network adapter;
921 is a RAM, 922 is a cache memory, 923 is a ROM, 924 is a program module, and 925 is a utility.

### DETAILED DESCRIPTION OF THE PREFERRED EMBODIMENT

A rating system of the present disclosure can be used to evaluate the severity of port-wine stains (PWS), the rating system comprising: an indicator selection and assignment module, an indicator processing module, and a rating module (see FIG. 1). Herein, the indicator selection and assignment module selects a rating indicator for a divided rating range and assigns a value to the indicator, the indicator processing module scores the severity of a port-wine stain focus, and the rating module rates the severity. Herein, the indicator processing module calls data in the indicator selection and assignment module to score the severity of the port-wine stain focus, followed by inputting the scoring result into the rating module, and the rating module outputs a rating result of the severity. The division of the rating range refers to dividing one or more areas to be examined on skin of a rating subject. Definition of terms are as follows.

### Definition of Terms

Rating subject: A collective term for patients and subjects who are to be rated on the severity of port-wine stains they have or may have in clinical treatment or clinical trials.

Rater: A collective term for physicians, specialists, and persons used to obtain data for control groups involved in rating the severity of the rating subjects in clinical treatment or clinical trials.

Focus: An area on the skin of a rating subject that presents or has presented symptoms of damage in port-wine stains (PWS).

Rating range: A range of skin to be rated for PWS severity, as defined in a given rating. In general, the rating range should be the same for all rating subjects in a clinical trial, at least in terms of area, so as to be comparable. The division of the rating range is also often related to the treatment and should preferably be no larger than the area of the skin that can be covered in a single treatment session. For example, topical medications typically have a smaller rating range, possibly on the face or hands; whereas oral medications can include the entire body of the patient in the rating range. If the affected part is so widely distributed that it cannot be completely covered in a single treatment session, it is recommended that the rating be done separately for a single treatment session.

Area to be examined: A geometrically continuous area that contains or may contain a focus of port-wine stains, defined within the rating range. Number of areas to be examined is ≥ 1, preferably from 1 to 10, for example 2, 3, 4, 5 or 6.

The difference between the area to be examined and the rating range is that the definition of the rating range is based on the purpose of rating without considering the accuracy and difficulty in rating; while the definition of the area to be examined is purely from the technical point of view in order to make the rating results more accurate.

The rating range may contain multiple areas to be examined that are continuous or discontinuous with each other. For example, if the rating range is a human face, then forehead, left and right cheeks, and chin may be defined as the areas to be examined, each of which may contain separate focuses. The rating results of each area to be examined at different locations are then summed according to the required weights to obtain a rating result of the face as a whole.

Multiple separate but continuous areas to be examined may also be defined within a large PWS focus which is continuous but particularly uneven in severity, such that all the areas to be examined are combined to cover the entire focus, while the focus in the part covered by each area to be examined is relatively homogeneous in severity. In this way, the rating results of each area to be examined will be more accurate. Finally, the rating results of all the areas to be examined are summed with equal weights, so as to obtain an overall rating result of the rating range including the entire focus.

Size: An area percentage of a site of the focus defined above in the area to be examined.

If the area of the site of the focus is large, or there is variation in symptom severity on one continuous focus, the focus may be divided into multiple sub-areas to be rated separately to improve the accuracy of the rating.

If the focuses are particularly small in area but numerous and scattered, they can also be combined to estimate their area by stitching

Hypertrophy: Symptoms of damage in PWS thicken with age, showing a change in texture. Therefore, the protruding degree of the skin surface of the site of the focus relative to the skin surface of a healthy site in the area to be examined is defined as hypertrophy.

Healthy color: The epidermal layer of human skin can have different colors due to pigmentation such as melanin, protoheme, and carotene. The so-called healthy color refers to the color of the epidermal layer of the skin in healthy sites of the rating subject. The healthy sites referred to here are sites where there is no hyperpigmentation caused by diseases other than PWS.

Color: Color, as defined herein, refers to the degree of color darkening of the epidermal layer of the site of the focus relative to the healthy color.

If the color of the site of the focus is uneven, the color of the darkest part is considered as the dominant part, and the color of the other parts is only used as a reference for the overall impression.

For different races, the shade of healthy color varies, so the color must be assigned with the difference in color between the epidermal layer of the site of the focus and the healthy sites.

Nodule: The present disclosure uses the most common clinical method of differentiating between papules and nodules, defining nodules as palpable localized and substantial skin lesions larger than 5 mm. They are often circular, oval, or irregular in shape.

Number of nodules: the number of nodules present in the site of the focus.

Location: Used to describe where the focus is located on the body of the rating subject.

Because PWS occurring in the face has the most serious negative impact on patients’ lives and the difficulty of treatment varies in different regions of the face, the body of the rating subject is often divided into two regions: head and neck, and trunk. In clinical practice, it is usually PWS occurring in the head and neck that causes the most distress to patients, so the head and neck can be further subdivided into a perifacial region (including frontal region, cheek region, temporal region, and parotid masseter region), a midfacial region (including orbital region, nasal region, infraorbital region, lip region, chin region, and zygomatic region), and a neck region (including anterior cervical region, lateral cervical region, and posterior cervical region). Alternatively, the face can also be divided into forehead, left cheek, right cheek and chin.

### Rating Methods

Selection and management of rating subjects:
The rating subjects includes untreated PWS patients, previously treated PWS patients, and control groups (healthy population).
The same rating range was defined for each rating subject.
Photos were taken of the areas to be examined for all rating subjects, which followed a unified process to ensure that light brightness and angle, camera parameters and other conditions were consistent. The photos were presented electronically on the display of a preselected computer to eliminate the possibility of color differences between different display devices. Each rating subject was photographed twice from two angles: front view and side view.

Selection and management of raters:
Raters were divided into experienced and inexperienced groups based on whether they had been involved in similar dermatologic ratings. The experienced group was primarily dermatology clinicians, and the inexperienced group was primarily non-clinical researchers and other staff.
All raters were given a unified learning session on the rating method, which lasted about one hour, mainly to explain the basis for the quantitative assignment of each indicator, and to teach how to fill in the corresponding printed scales and computer software.

Specific rating process:
The ratings are divided into face-to-face diagnosis ratings and photo ratings. In face-to-face diagnosis ratings, raters were allowed to face the rating subjects directly and assign quantitative values by observation and palpation; in photo ratings, raters were only allowed to see the electronic photos and assign quantitative values by visual alone.
The raters were asked to enter the room of each rating subject in a random order (face-to-face diagnosis rating) or to browse the photos of each rating subject (photo rating), and to assign quantitative values to five indicators of color, size, hypertrophy, number of nodules and location of the rating subject within a specified time (the experiment stipulated that the rating time for each rating subject should not exceed 5 minutes), and to fill assigned data into statistical tables in computer.
The data in the statistical tables of all the raters were calculated in an automatic way by computer software or manually to obtain the rating results, according to the formulas in the method of the present disclosure.

The embodiments of the present disclosure provide several calculation schemes with different emphases, and the advantages and disadvantages of each formula are summarized below based on their respective rating results.

The present disclosure is further illustrated below by means of embodiments, but the present disclosure is not thereby limited to the scope of the embodiments. Experimental methods for which specific conditions are not specified in the following embodiments are selected according to conventional methods and conditions, or according to the trade description.

### Embodiment 1: Indicators of Skin Are Color, Hypertrophy and Size (formula 1)

The area to be examined was defined as a circular area with a diameter of 10 cm.

The assignment was performed by dividing color into 5 categories, hypertrophy into 4 gradients, and size into 5 intervals, that is, C = 5, H = 4, and S = 5, as shown in Table 1.

The formula for the quantitative calculation is X₁= (Cᵥ + Hᵥ) × Sᵥ.

TABLE 1

Quantitative assignment of each indicator in Embodiment 1

| Indicators | Assigned values | | | | | Name of quantitative assignment |
|---|---|---|---|---|---|---|
| Color (C) | 0 | 1 | 2 | 3 | 4 | Category |
| Hypertrophy (H) | 0 | 1 | 2 | 3 | / | Gradient |
| Size (S) | 0 | 1 | 2 | 3 | 4 | Interval |

### Embodiment 2: Indicators of Skin Are Color, Hypertrophy and Size (formula 2)

The area to be examined was defined as a circular area with a diameter of 10 cm.

The assignment was performed by dividing color into 5 categories, hypertrophy into 4 gradients, and size into 5 intervals, that is, C = 5, H = 4, and S = 5, as shown in Table 1.

The formula for the quantitative calculation is X₂ = 0.7 × Cᵥ + 0.2 × Hᵥ + 0.1 × Sᵥ.

### Evaluation Embodiment

Embodiment 1 uses the calculation formula of formula 1, Embodiment 2 uses the calculation formula of formula 2, and the calculation results are shown in Table 2.

TABLE 2

Quantitative rating results and corresponding abstraction grading results

| X₁ | X₂ | Level | PWS severity |
|---|---|---|---|
| 0-5 | 0-0.76 | 0 | Normal |
| 6-11 | 0.77-1.52 | 1 | Mild |
| 12-17 | 1.53-2.28 | 2 | Moderate |
| 18-23 | 2.29-3.04 | 3 | Severe |
| 24-28 | 3.05-3.80 | 4 | Extremely severe |

For the three indicators of color, hypertrophy and size, in addition to the above calculation formulas, weights may be adjusted according to clinical needs to establish a more suitable calculation formula.

For example, when focusing on the efficacy of eliminating hypertrophy, the weight of color may be reduced and the weight of hypertrophy may be increased; and vice versa.

Also, for light-skinned people, the color of the site of the focus is the most influential indicator of appearance, so the weight of color may be increased; and vice versa.

### Embodiment 3: A Scheme That Further Takes Indicators of Number of Nodules And location into consideration

After calculating the quantitative rating value of an area to be examined according to the method in Embodiment 1 or 2, the calculation result of the quantitative rating may be further adjusted by the location of the focus or the number of nodules contained therein.

If the focus is located in the midfacial region, a value of 1.3 is assigned to location L; if the focus is located in the perifacial region and neck region, a value of 1.1 is assigned to location L. Adjusted X₁’ = X₁ × Lᵥ; X₂’ = X₂ × Lᵥ. The level may be later obtained by abstraction grading based on the values of X₁’ and X₂′.

For the results of abstraction grading, the number of nodules of the site of the focus is further examined. The number of nodules is divided into N groups based on the number of palpable localized and substantial skin focuses, N ≥ 2; preferably from 2 to 4. For example, the number of nodules is divided into 3 groups: group 0 contains no nodules, Nᵥ = 0; group 1 contains 1-5 nodules, Nᵥ = 1; group 2 contains at least 6 nodules, Nᵥ = 2. Thus, the indicator of number of nodules was introduced as Level′ = Level + Nᵥ.

### Embodiment 4: A Scheme for Defining a Face as a Whole as the Rating Range

In this embodiment, four indicators of color, hypertrophy, size, and location are taken into consideration simultaneously, and the defined rating range is the face.

### (1) Quantitative Assignment Scheme:

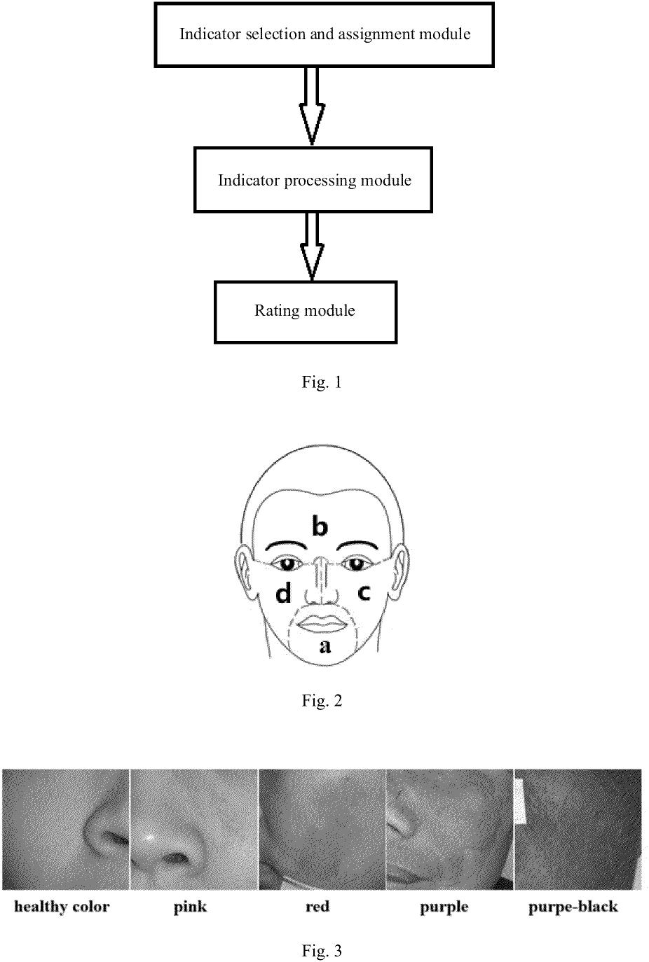

Four areas to be examined are defined on the face as shown in FIG. 2, namely: chin, forehead, left cheek and right cheek, marked as a, b, c and d, respectively.

As shown in Table 3, the four indicators of color, hypertrophy, size, and location are assigned as follows.

The location is divided into 3 grades (i.e., L = 3). Grade 0 represents no PWS focuses on the face, Lᵥ = 0; grade 1 represents a PWS focus located in the area to be examined a (chin), Lᵥ = 0.1; grade 2 represents a focus located in the area to be examined b (forehead) or the area to be examined c (left cheek) or the area to be examined d (right cheek), Lᵥ = 0.3.

The color is divided into 5 categories (i.e., C = 4). Cᵥ = 0 represents healthy color; Cᵥ = 1 represents flesh pink; Cᵥ = 2 represents red; Cᵥ = 3 represents purple, and Cᵥ = 4 represents dark purple. It is noted that the case in this embodiment is yellow race, so the healthy color is based on the general color of the yellow race, and the color standards for subsequent categories are also derived from clinical trial data of the yellow race cases. For application to clinical trials with other races, the baseline of healthy color needs to be replaced, and the color standards for subsequent categories should be changed accordingly.

The size is divided into 7 intervals (i.e., S = 7). Interval 0 (i.e., Sᵥ = 0) represents no PWS focuses on the face; interval 1 (i.e., Sᵥ = 1) represents the proportion of the focus is <10%; interval 2 (i.e., Sᵥ = 2) represents the proportion of the focus is 10-29%; interval 3 (i.e., Sᵥ = 3) represents the proportion of the focus is 30-49%; interval 4 (i.e., Sᵥ = 4) represents the proportion of the focus is 50-69%; interval 5 (i.e., Sᵥ = 5) represents the proportion of the focus is 70-89%; interval 6 (i.e., Sᵥ = 6) represents the proportion of the focus is 90-100%. It is noted that the division of intervals for size in this embodiment is uneven, with interval 1 and interval 6 (i.e., the least and the most severe intervals other than the healthy interval 0) accounting for a smaller proportion.

The hypertrophy is divided into 4 gradients (i.e., H = 4). Gradient 0 (i.e., Hᵥ = 0) means that the face is completely flat; gradient 1 (i.e., Hᵥ = 1) means that the focus is slightly protruded; gradient 2 (i.e., Hᵥ = 2) means that the focus is significantly protruded; gradient 3 (i.e., Hᵥ = 3) means that the focus is significantly protruded and accompanied by nodules.

TABLE 3

Quantitative assignment of each indicator in Embodiment 4

| Indicators | Assigned values | | | | | | | Name of quantitative assignment |
|---|---|---|---|---|---|---|---|---|
| L | 0 | 0.1 | 0.3 | / | / | / | / | Grade |
| C | 0 | 1 | 2 | 3 | / | / | / | Category |
| S | 0 | 1 | 2 | 3 | 4 | 5 | 6 | Interval |
| H | 0 | 1 | 2 | 3 | / | / | / | Gradient |

For the area to be examined a, the calculation scheme is:

$${\text{X}}_{\text{3a}} = {\text{L}}_{\text{v-a}} \times {\text{S}}_{\text{v-a}} \times \left({\text{C}}_{\text{v-a}} + {\text{H}}_{\text{v-a}}\right)$$

In this embodiment, the indicator of number of nodules is essentially included in the hypertrophy indicator, so as to reduce the complexity of the calculation. If the number of nodules is desired to calculate by splitting, gradient 2 and gradient 3 may be combined so that the hypertrophy H is only divided into 3 gradients, and the subsequent calculations remain unchanged; then the number of nodules is assigned a value of 0 or 1, following the rule that the number of nodules is assigned a value of 1 as long as there are nodules regardless of the specific number of nodules. Then the calculation scheme is modified as follows:

$${\text{X}}_{3 \text{a}} \text{} ′ = {\text{L}}_{\text{v-a}} \times {\text{S}}_{\text{v-a}} \times \left({\text{C}}_{\text{v-a}} + {\text{H}}_{\text{v-a}} + {\text{N}}_{\text{v-a}}\right)$$

The result remains the same whether or not the number of nodes is calculated by splitting.

The same calculation scheme is used for the areas to be examined b, c and d, and X_{3b}, X_{3c} and X_{3d} were obtained respectively.

The rating results of the four areas to be examined are finally summed to obtain a quantitative rating result of the face as a whole:

$${\text{X}}_{\text{3}} = {\text{X}}_{\text{3a}} + {\text{X}}_{\text{3b}} + {\text{X}}_{\text{3c}} + {\text{X}}_{\text{3d}}$$

In this embodiment, after calculating the quantitative rating result X₃ according to the above scheme, there is no need to further grade X₃, but to directly use the value of X₃ for the efficacy evaluation. For the same rating subject, the severity of facial PWS was rated quantitatively before and after treatment to obtain X₃₁ and X₃₂ respectively, and the quantitative value of efficacy may be obtained by calculating X₃₁ - X₃₂, thereby achieving the highest accuracy of efficacy evaluation.

### (2) The Advantages and Disadvantages of the Scheme in Embodiment 4 Were Examined in a statistical manner:

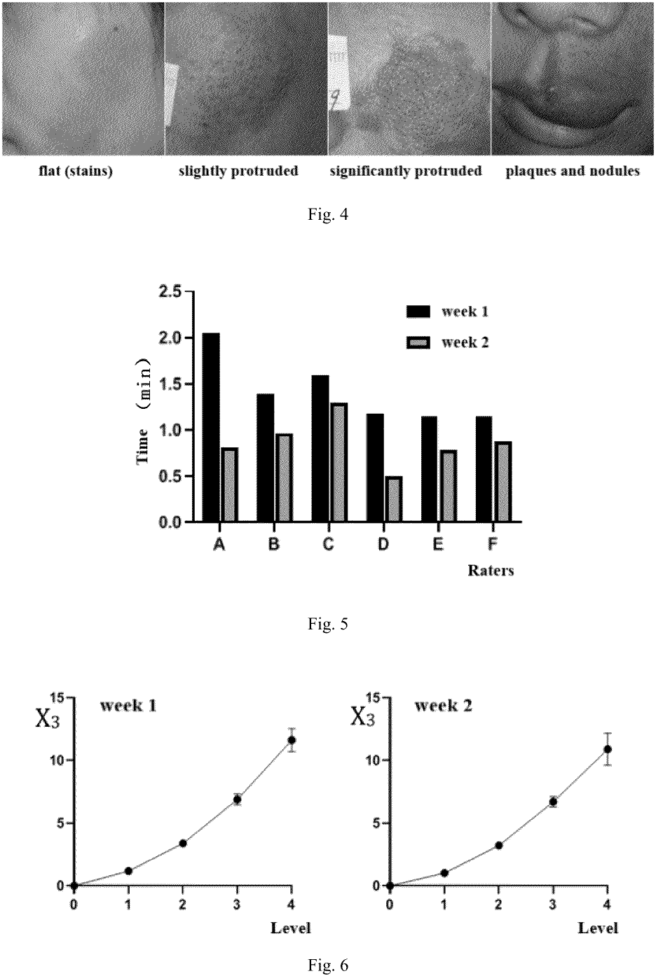

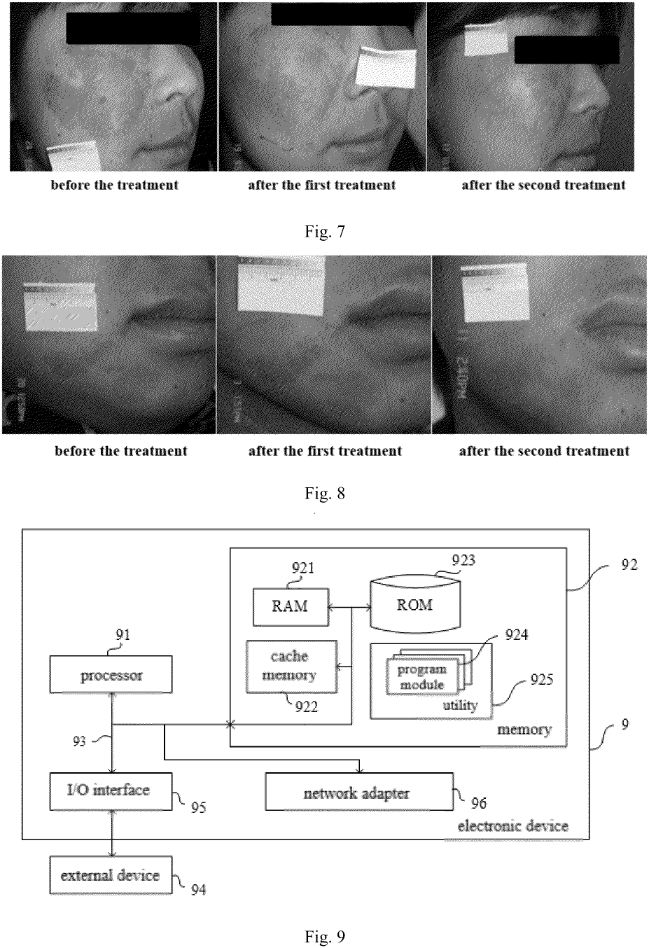

FIG. 5 shows a comparison of the time required to evaluate 1 photo (i.e., 1 area to be examined) by 6 raters who rated the photos (classification as shown in FIG. 3 and FIG. 4, and the photos as shown in FIG. 7 and FIG. 8) on two successive occasions in week 1 and week 2. It can be seen that the raters can quickly master the rating method of the present disclosure, and the time required was significantly reduced. And the difference between the 6 raters was very small, statistically p = 0.018 < 0.05.

Similarly, the reliability of the scheme in Embodiment 4 was examined based on the rating data from the above 6 raters on two successive occasions. The parameters examined include: external differences between different raters (inter-rater reliability), and internal differences for the same rater due to the presence or absence of experience (intra-rater reliability), the results are shown in Tables 4-7.

TABLE 4

External differences between different raters

| | Intraclass correlation coefficient (r) |
|---|---|
| Week 1 | 0.856 |
| Week 2 | 0.810 |

TABLE 5

Internal differences between weeks for the same rater

| | Intraclass correlation coefficient (r) |
|---|---|
| Rater 1 | 0.913 |
| Rater 2 | 0.929 |
| Rater 3 | 0.950 |
| Rater 4 | 0.861 |
| Rater 5 | 0.928 |
| Rater 6 | 0.917 |

TABLE 6

Internal differences between weeks for assignments of size, assignments of color, and assignments of hypertrophy (weighted Kappa coefficients)

| Indicators for different locations | Kappa | | | | | | |
|---|---|---|---|---|---|---|---|
| | Rater 1 | Rater 2 | Rater 3 | Rater 4 | Rater 5 | Rater 6 | Average |
| Sb | 0.871 | 0.853 | 0.840 | 0.612 | 0.855 | 0.873 | 0.817 |
| Cb | 0.712 | 0.815 | 0.866 | 0.572 | 0.872 | 0.825 | 0.777 |
| Hb | 0.622 | 0.622 | 0.821 | 0.585 | 0.623 | 0.764 | 0.684 |
| Sd | 0.87 | 0.889 | 0.922 | 0.776 | 0.849 | 0.899 | 0.868 |
| Cd | 0.823 | 0.869 | 0.890 | 0.757 | 0.813 | 0.827 | 0.830 |
| Hd | 0.765 | 0.745 | 0.805 | 0.781 | 0.739 | 0.733 | 0.761 |
| Sc | 0.854 | 0.887 | 0.929 | 0.823 | 0.855 | 0.912 | 0.877 |
| Cc | 0.774 | 0.812 | 0.905 | 0.680 | 0.787 | 0.854 | 0.802 |
| Hc | 0.742 | 0.599 | 0.835 | 0.814 | 0.669 | 0.765 | 0.737 |
| Sa | 0.770 | 0.809 | 0.904 | 0.714 | 0.859 | 0.852 | 0.818 |
| Ca | 0.716 | 0.772 | 0.904 | 0.542 | 0.791 | 0.846 | 0.762 |
| Ha | 0.638 | 0.512 | 0.809 | 0.639 | 0.713 | 0.745 | 0.676 |

(Kappa benchmark: 0.00 = poor; 0.0-0.2 = slightly approximate; 0.2-0.4 = average; 0.4-0.6 = moderate; 0.6-0.8 = basically consistent; 0.8-1.0 = nearly perfect)

TABLE 7

External differences between weeks for assignments of size, assignments of color, and assignments of hypertrophy (Kendall coordination coefficient W test)

| Indicators for different locations | Kendall coordination coefficient W | |
|---|---|---|
| | Week 1 | Week 2 |
| Sb | 0.850 | 0.877 |
| Cb | 0.862 | 0.870 |
| Hb | 0.622 | 0.622 |
| Sd | 0.929 | 0.935 |
| Cd | 0.898 | 0.918 |
| Hd | 0.799 | 0.805 |
| Sc | 0.941 | 0.928 |
| Cc | 0.911 | 0.903 |
| Hc | 0.784 | 0.769 |
| Sa | 0.813 | 0.872 |
| Ca | 0.757 | 0.854 |
| Ha | 0.666 | 0.712 |

The validity of the scheme in Embodiment 4 was next examined.

As a measurement standard, it is required to first establish a rating system (Physician global assessment (PGA)) that will be assigned to clinical experts. The grading of this system is consistent with that of Embodiment 4, in which PWS is divided into 5 levels from mild to severe: level 0 = normal; level 1 = showing flat pink = mild; level 2 = showing flat red = moderate; level 3 = showing protruded purple = severe; level 4 = showing significant protruded purple-black with nodules = extremely severe.

For each rating subject, the rating is first performed by the experienced clinical expert following the PGA system to obtain an accurate level value as the measurement standard. The quantitative assignment and calculation is then performed by the rater following the scheme in Embodiment 4 to obtain X₃.

Taking X₃ (95% confidence interval of the mean) as the vertical axis and level as the horizontal axis, the results are shown in FIG. 6. X₃ increases with the increase of level with a linear trend of P < 0.0001.

TABLE 8

Correlation between X₃ and level (Spearman’s rank correlation coefficient)

| Raters | Week 1 | Week 2 |
|---|---|---|
| Rater 1 | 0.847 | 0.805 |
| Rater 2 | 0.863 | 0.870 |
| Rater 3 | 0.820 | 0.789 |
| Rater 4 | 0.752 | 0.761 |
| Rater 5 | 0.907 | 0.905 |
| Rater 6 | 0.785 | 0.753 |

As can be seen from the above analysis, the rating conclusions reached by multiple raters following the scheme in Embodiment 4 are highly consistent with those reached by experienced clinical experts, proving that the rating scheme of the present disclosure can indeed serve to help general physicians and related practitioners to quickly obtain highly accurate rating results, which is conducive to extensively improving the level of diagnosis and treatment of PWS in various hospitals.

### (3) An Auxiliary Tool

The rating system of the present disclosure further comprises an auxiliary tool for quantifying and assigning values to each indicator, such as a colorimetric standard for quantifying and assigning values to the indicator of the color, or a measuring tool for quantifying and assigning values to the indicator of the size. The colorimetric standard may be a flat print or a spherical three-dimensional print; for example, a flat print of a paper color card. At least D color blocks are printed sequentially on the colorimetric standard according to the color from light to dark, the color of each of the categories is the same as color of at least one color block, the color blocks are, for example, circular or apple-shaped. The measuring tool is a flat print of the same shape as the area to be examined, on which checkerboard-shaped square grids of the same size are printed, such as a grid paper, or a soft transparent or translucent material.

### Embodiment 5: Clinical Efficacy Evaluation Using the Scheme in Embodiment 4

Clinical efficacy evaluation scheme: The rating subjects were divided into an experimental group and a control group with equal numbers, and each group was treated twice. The experimental group was treated with drugs on both occasions; the control group was treated with placebo for the first time and with drugs for the second time.

At three time points, before the first treatment, after the first treatment, and after the second treatment, the overall facial PWS severity of the rating subjects was rated using the scheme in Embodiment 4, and the quantitative rating result X₃ for each rating subject was calculated to provide an accurate evaluation of the efficacy based on the data.

### Embodiment 6: Electronic Device

This embodiment provides an electronic device that can be represented in the form of a computing device (which may be, for example, a server device), comprising a memory, a processor, and a computer program stored in the memory and operable on the processor, wherein the processor, when executing the computer program, may implement the rating methods in Embodiments 1-4 of the present disclosure.

As shown in FIG. 9, an electronic device 9 specifically includes:

at least one processor 91, at least one memory 92, and a bus 93 for connecting different system components (including the processor 91 and the memory 92), wherein:

the bus 93 includes a data bus, an address bus, and a control bus.

The memory 92 includes a volatile memory, such as a random access memory (RAM) 921 and/or a cache memory 922, and may further include a read-only memory (ROM) 923.

The memory 92 also includes a program/utility 925 having a set of (at least one) program modules 924, the program modules 924 including, but not limited to, an operating system, one or more application programs, other program modules and program data, each of these examples or some combination thereof may include an implementation of a network environment.

The processor 91 executes various functional applications as well as data processing, such as the rating methods in Embodiments 1-4 of the present disclosure, by running a computer program stored in the memory 92.

The electronic device 9 may further communicate with one or more external devices 94 (e.g., keyboards, pointing devices, etc.). This communication may be done through an input/output (I/O) interface 95. Moreover, the electronic device 9 may also communicate with one or more networks (e.g., a local area network (LAN), a wide area network (WAN), and/or a public network, such as the Internet) via a network adapter 96. The network adapter 96 communicates with other modules of the electronic device 9 via bus 93. It should be understood that although not shown in the figures, other hardware and/or software modules may be used in conjunction with the electronic device 9, including but not limited to: microcode, a device driver, a redundant processor, an external disk drive array, a RAID (Redundant Arrays Of Independent Disks) system, a tape drive, and a data backup storage system.

The external device connected to the electronic device 9 also includes a printer, so that the results can be printed using the printer (result printing module) after the evaluation to obtain the rating results output by the rating module. In addition, before the rating, the input image result can be provided to the indicator selection and assignment module using communication means such as a keyboard or an influence device (input module) in the external device 94 described above. The image result is a face-to-face diagnosis result or a photo result.

It should be noted that although a number of units/modules or subunits/modules of the electronic device are mentioned in the detailed description above, such division is only exemplary and not mandatory. In fact, according to the embodiments of the present disclosure, the features and functions of two or more units/modules described above may be embodied in one unit/module. Conversely, the features and functions of one unit/module described above can be further divided to be embodied by a plurality of units/modules.

### Embodiment 7: Computer-readable Storage Medium

This embodiment provides a computer-readable storage medium on which a computer program is stored, the program implements the steps of the rating methods in Embodiments 1-4 when executed by a processor.

Herein, the readable storage medium may more specifically include but not limited to: a portable disk, a hard disk, a random access memory, a read-only memory, an erasable programmable read-only memory, an optical storage device, a magnetic storage device, or any suitable combination of the above.

In possible embodiments, the present disclosure may also be implemented in the form of a program product comprising a program code, when the program product runs on a terminal device, the program code is used to cause the terminal device to implement the steps of the rating methods in Embodiments 1-4 of the present disclosure.

Herein, the program code for executing the present disclosure may be written in any combination of one or more programming languages. The program code may be executed entirely on the user device, partially on the user device, as a stand-alone software package, partially on the user device and partially on a remote device, or entirely on the remote device.

Although the specific embodiments of the present disclosure have been described above, it should be understood by those skilled in the art that these are merely illustrative examples and that a variety of changes or modifications can be made to these embodiments without departing from the principles and substance of the present disclosure. Therefore, the scope of protection of the present disclosure is limited by the appended claims. It is intuitively evident that during the treatment, the change in the severity of the focus of the rating subject is consistent with the change in the rating result X₃, thus demonstrating the clinical significance of the rating scheme of the present disclosure.

What is claimed is:

1. A severity rating system for port-wine stains comprising an indicator selection and assignment module, an indicator processing module, and a rating module, wherein: the indicator selection and assignment module selects a rating indicator for a divided rating range and assigns a value to the indicator, the indicator processing module scores the severity of a port-wine stain focus, and the rating module rates the severity; wherein, the indicator processing module calls data in the indicator selection and assignment module to score the severity of the port-wine stain focus, followed by inputing inputting the scoring result into the rating module, and the rating module outputs a rating result of the severity;
the division of the rating range refers to dividing one or more areas to be examined on skin of a rating subject, and the area to be examined is an area that is geometrically contiguous without overlapping and that contain or may contain a focus of a port-wine stain.

2. The rating system of claim 1, wherein, the rating indicator in the indicator selection and assignment module comprises a size and a hypertrophy;
the size refers to an area percentage of the focus in the area to be examined;
the hypertrophy refers to a protruding degree of a skin surface of the focus relative to a skin surface of a healthy site in the area to be examined;
preferably, the rating indicator further comprises a color;
the color refers to a degree of color darkening of an epidermal layer of a site of the focus relative to an epidermal layer of the healthy site in the area to be examined;
more preferably, the rating indicator further comprises a number of nodules and/or a location;
the number of nodules refers to number of palpable localized and substantial skin lesions;
the location refers to a site where the focus is located on the body of the rating subject.

3. The rating system of claim 1, wherein, in the indicator selection and assignment module, assigning a value to the rating indicator refers to assigning a value to selected indicators in accordance with an assignment method, and the assignment method comprises:
dividing the size into S intervals in order from small to large according to a percentage of the focus in the area to be examined, wherein S ≥ 2; preferably 2 to 7; more preferably 4 to 6; when a value of the interval Sᵥ = 0, the percentage of the interval contains 0 and there is no clinically significant focus; when Sᵥ is a maximum value, the percentage of the interval contains 100%, that is, a site of the focus fully occupies the area to be examined;
dividing the hypertrophy into H gradients according to a visually identifiable protruding degree of a skin surface of the focus relative to a skin surface of a healthy site of the area to be examined, H ≥ 2; preferably 2 to 4; when a value of the gradient Hᵥ = 0, the protruding degree is 0, when Hᵥ is a maximum value, the protruding degree is a marked protrusion;
preferably, the color is divided into C categories in order from light to dark according to a degree of color darkening of an epidermal layer of the focus, C ≥ 2; preferably 4 to 6; when a value of the category Cᵥ = 0, the color is a healthy color, when Cᵥ is a maximum value, the color is purple-black;
more preferably, the number of nodules is divided into N groups according to the number of palpable localized and substantial skin lesions, N ≥ 2; preferably 2 to 4, for example N = 3; when a value of the number Nᵥ = 0, there are no nodules, when Nᵥ is a maximum value, the number of nodules is greater than 10; and/or,
dividing the location into L grades according to the site where the focus is located on the body of the rating subject, L ≥ 2; preferably 2 to 6, more preferably 3 to 4; when a value of the grade Lᵥ = 0, the focus is located below the neck, when Lᵥ is a maximum value, the focus is located in middle of a face.

4. The rating system of claim 3, wherein, in the indicator selection and assignment module,
when S = 5, Sᵥ ranges from 0 to 4, when Sᵥ = 0, the percentage is 0-20%; when Sᵥ = 1, the percentage is 20-40%; when Sᵥ = 2, the percentage is 40-60%; when Sᵥ = 3, the percentage is 60-80%; when Sᵥ = 4, the percentage is 80-100%;
when H = 4, Hᵥ ranges from 0 to 3, Hᵥ = 1 means a degree of protrusion that is difficult to be visually identified but palpable by palpation; Hᵥ = 2 means a little protrusion that is visually identifiable; Hᵥ = 3 means a marked protrusion that is visually identifiable;
preferably, when C = 5, Cᵥ ranges from 0 to 4, when Cᵥ = 0, the color is a healthy color; when Cᵥ = 1, the color is pink that is slightly darker than a color of the healthy site; when Cᵥ = 2, the color is red; when Cᵥ = 3, the color is purple; when Cᵥ = 4, the color is purple-black;
more preferably, when L = 4, Lᵥ ranges from 1.0 to 1.3, Lᵥ = 1.0 means that the focus is located at a site below a neck; Lᵥ = 1.1 means that the focus is located in the neck; Lᵥ = 1.2 means that the focus is located in periphery of the face; Lᵥ = 1.3 means that the focus is located in the middle of the face; and/or,
when L = 3, a range of Lᵥ is (0, 0.1, 0.3), Lᵥ = 0 means that the focus is located on a non-face site; Lᵥ = 0.1 means that the focus is located on a chin; Lᵥ = 0.3 means that the focus is located on a forehead or a left cheek or a right cheek.

5. The rating system of claim 3, wherein, in the indicator processing module, scoring the severity refers to: performing a quantitative calculation and weighting on each area to be examined according to weight and assigned value of each indicator of the area to be examined to obtain a score of the severity of the rating range;
preferably, a formula for the quantitative calculation is as follows:
(1) when weights of the selected indicators are size, color and hypertrophy in descending order:
$${\text{the score of the severity X}}_{1} = \left({\text{C}}_{\text{v}} + {\text{H}}_{\text{v}}\right) \times {\text{S}}_{\text{v}} \qquad \text{(formula 1)}$$
wherein, the severity is the a lowest level when X
₁ = 0; the severity is a highest level when X₁ = [(C - 1) + (H - 1)] × (S - 1);
(2) when the weights of the selected indicators are color, hypertrophy and size in descending order,
$${\text{the score of the severityX}}_{2} = 0.7 \times {\text{C}}_{\text{v}} + 0.2 \times {\text{H}}_{\text{v}} + 0.1 \times {\text{S}}_{\text{v}} \qquad \text{(formula 2)}$$
wherein, the severity is the lowest level when X
₂ = 0; the severity is the highest level whenX₂ = (0.7 × C + 0.2 × H + 0.1 × S) - 1;
more preferably, when the selected indicator further comprises location:
the score of the severityX₁′ = X₁ × Lᵥ;
the score of the severity X₂′ = X₂ × Lᵥ;
further more preferably, when the selected indicator further comprises the number of nodules:
$${\text{the score of the severity X}}_{3} = {\text{L}}_{\text{v}} \times {\text{S}}_{\text{v}} \times \left({\text{C}}_{\text{v}} + {\text{H}}_{\text{v}} + {\text{N}}_{\text{v}}\right) .$$
.

6. The rating system of claim 5, wherein, in the rating module, when S = 5, H = 4, C = 5, the severity of the focus is classified into five successive and independent levels from mild to severe;
when X₁ = 0-5, the severity of the site of the focus is the lowest level; when X₁ = 6-11, the severity of the site of the focus is a second lowest level; when X₁ = 12-17, the severity of the site of the focus is an intermediate level; when X₁ = 18-23, the severity of the site of the focus is a second highest level; when X₁ = 24-28, the severity of the site of the focus is the highest level; or
when X₂ = 0-0.76, the severity of the site of the focus is the lowest level; when X₂ = 0.77-1.52, the severity of the site of the focus is the second lowest level; when X₂ = 1.53-2.28, the severity of the site of the focus is the intermediate level; when X₂ = 2.29-3.04, the severity of the site of the focus is the second highest level; when X₂ = 3.05-3.80, the severity of the site of the focus is the highest level.

7. The rating system of claim 1, wherein, number of areas to be examined is ≥ 1, preferably 1 to 10, for example 2, 3, 4, 5 or 6;
and/or, a geometry of the area to be examined comprises a square, a rectangle or a circle;
and/or, the rating range is pre-defined prior to the division of the rating ranges; the area to be examined is preferably comprised in the rating range; the rating range more preferably comprises a face or a whole body.

8. The rating system of claim 1, wherein, the rating system further comprises a result printing module to print the rating result output by the rating module;
and/or, the rating system an input module, providing an input image result to the indicator selection and assignment module; preferably, the image result is a face-to-face diagnosis result or a photo result.

9. The rating system of claim 1, wherein, the rating system further comprises an auxiliary tool for quantifying and assigning values to each indicator;
preferably, the auxiliary tool is a colorimetric standard for quantifying and assigning values to the indicator of the color; and/or, the auxiliary tool is a measuring tool for quantifying and assigning values to the indicator of the size;
more preferably, the colorimetric standard is a flat print or a spherical three-dimensional print; for example, a flat print of a paper color card; and/or, the measuring tool is a flat print of the same shape as the area to be examined, on which checkerboard-shaped square grids of the same size are printed, such as a grid paper;
further more preferably, at least D color blocks are printed sequentially on the colorimetric standard according to the color from light to dark, the color of each of the categories is the same as color of at least one color block; the color block is preferably circular or apple-shaped; and/or, the measuring tool is a soft transparent or translucent material.

10. A rating method, comprising:
P1: defining a rating range comprising one or more areas to be examined on skin of a rating subject, and the area to be examined is an area that is geometrically contiguous without overlapping and that contain or may contain a focus of port-wine stains;
P2: performing a numerical quantitative assignment of at least one indicator of the skin of a site of the focus of the port-wine stains defined in P1 by calling an indicator selection and assignment module;
P3: calculating, by an indicator processing module, numerical rating results of the severity of the port-wine stains in the area to be examined based on results of the quantitative assignment of indicators of the skin in P2, and outputting the numerical rating results through a rating module;
preferably, number of areas to be examined is ≥ 1, preferably 1 to 10, for example 2, 3, 4, 5 or 6; and/or,
a geometry of the area to be examined comprises a square, a rectangle or a circle; and/or,
the rating range comprises a face or a whole body.

11. The rating method of claim 10, wherein, the indicator in the step P2 comprises: a size and a hypertrophy;
preferably, the size refers to an area percentage of the focus in the area to be examined; the hypertrophy refers to a protruding degree of a skin surface of the focus relative to a skin surface of a healthy site in the area to be examined; and/or, the indicator further comprises a color;
more preferably, the color refers to a degree of color darkening of an epidermal layer of a site of the focus relative to an epidermal layer of the healthy site in the area to be examined; and/or, the indicator further comprises number of nodules and/or a location;
further more preferably, the number of nodules refers to number of palpable localized and substantial skin lesions; and/or, the location refers to a site where the focus is located on the body of the rating subject.

12. The rating method of claim 11, wherein, the quantitative assignment of the indicators refers to assigning values to selected indicators in accordance with an assignment method; the assignment method specifically comprises:
dividing the size into S intervals in order from small to large according to a percentage of the focus in the area to be examined, S ≥ 2; preferably 2 to 7; more preferably 4 to 6; when a value of the interval Sᵥ = 0, the percentage of the interval contains 0 and there is no clinically significant focus; when Sᵥ is a maximum value, the percentage of the interval contains 100%, that is, the site of the focus fully occupies the area to be examined;
dividing the hypertrophy into H gradients according to a visually identifiable protruding degree of the skin surface of the focus relative to the skin surface of the healthy site of the area to be examined, H ≥ 2; preferably 2 to 4; when a value of the gradient Hᵥ = 0, the protruding degree is 0, when Hᵥ is a maximum value, the protruding degree is a marked protrusion;
preferably, the color is divided into C categories in order from light to dark according to the degree of color darkening of the epidermal layer of the site of the focus, C ≥ 2; preferably 4 to 6; when a value of the category Cᵥ = 0, the color is a healthy color, when Cᵥ is a maximum value, the color is purple-black;
more preferably, the number of nodules is divided into N groups according to the number of palpable localized and substantial skin lesions, N ≥ 2; preferably 2 to 4, for example N = 3; when a value of the number Nᵥ = 0, there are no nodules, when Nᵥ is a maximum value, the number of nodules is greater than 10; and/or,
dividing the location into L grades according to the site where the focus is located on the body of the rating subject, L ≥ 1; preferably 1 to 1.3, for example 1.1, 1.2 or 1.3; when a value of the grade Lᵥ = 0, the focus is located below the neck, when Lᵥ is a maximum value, the focus is located in middle of the face.

13. The rating method of claim 12, wherein,
when S = 5, Sᵥ ranges from 0 to 4, when Sᵥ = 0, the percentage is 0-20%; when Sᵥ = 1, the percentage is 20-40%; when Sᵥ = 2, the percentage is 40-60%; when Sᵥ = 3, the percentage is 60-80%; when Sᵥ = 4, the percentage is 80-100%;
when H = 4, Hᵥ ranges from 0 to 3, Hᵥ = 1 means a degree of protrusion that is not visually identifiable but palpable by palpation; Hᵥ = 2 means a little protrusion that is visually identifiable; Hᵥ = 3 means a marked protrusion that is visually identifiable;
preferably, when C = 5, Cᵥ ranges from 0 to 4, when Cᵥ = 0, the color is a healthy color; when Cᵥ = 1, the color is pink that is slightly darker than a color of the healthy site; when Cᵥ = 2, the color is red; when Cᵥ = 3, the color is purple; when Cᵥ = 4, the color is purple-black;
more preferably, when L = 4, Lᵥ ranges from 1.0 to 1.3, Lᵥ = 1.0 means that the focus is located below the neck; Lᵥ = 1.1 means that the focus is located in the neck; Lᵥ = 1.2 means that the focus is located in the periphery of the face; Lᵥ = 1.3 means that the focus is located in the middle of the face.

14. The rating method of claim 10, wherein, calculating the numerical rating results of the severity in the step P3 refers to: performing a quantitative calculation and weighting on each area to be examined according to weight and assigned value of each indicator of the area to be examined to obtain a score of the severity of the rating range;
preferably, the formula for the quantitative calculation is as follows:
(1) when weights of the selected indicators are size, color and hypertrophy in descending order:
$${\text{the score of the severity X}}_{1} = \left({\text{C}}_{\text{v}} + {\text{H}}_{\text{v}}\right) \times {\text{S}}_{\text{v}} \qquad \text{(formula 1)}$$
wherein, the severity is a lowest level when X
₁ = 0; the severity is a highest level when X₁ = [(C - 1) + (H - 1)] × (S - 1);
(2) when the weights of the selected indicators are color, hypertrophy and size in descending order:
$${\text{the score of the severity X}}_{2} = 0.7 \times {\text{C}}_{\text{v}} + 0.2 \times {\text{H}}_{\text{v}} + 0.1 \times {\text{S}}_{\text{v}} \qquad \text{(formula 2)}$$
wherein, the severity is the lowest level when X
₂ = 0; the severity is the highest level when X₂ = (0.7 × C + 0.2 × H + 0.1 × S) - 1;
when the selected indicator further comprises the location:
$${\text{the score of the severity X}}_{1} {}^{′} = {\text{X}}_{1} \times {\text{L}}_{\text{v}} ;$$
$${\text{the score of the severity X}}_{2} {}^{′} = {\text{X}}_{2} \times {\text{L}}_{\text{v}} ;$$
when the selected indicator further comprises the number of nodules:
$${\text{the score of the severity X}}_{3} = {\text{L}}_{\text{v}} \times {\text{S}}_{\text{v}} \times \left({\text{C}}_{\text{v}} + {\text{H}}_{\text{v}} + {\text{N}}_{\text{v}}\right) ;$$
more preferably, when C = 5, S = 5, H = 4, the severity of the focus is classified into five successive and independent levels from mild to severe;
when X₁ = 0-5, the severity of the site of the focus is the lowest level; when X₁ = 6-11, the severity of the site of the focus is a second lowest level; when X₁ = 12-17, the severity of the site of the focus is an intermediate level; when X₁ = 18-23, the severity of the site of the focus is a second highest level; when X₁ = 24-28, the severity of the site of the focus is the highest level; or
when X₂ = 0-0.76, the severity of the site of the focus is the lowest level; when X₂ = 0.77-1.52, the severity of the site of the focus is the second lowest level; when X₂ = 1.53-2.28, the severity of the site of the focus is the intermediate level; when X₂ = 2.29-3.04, the severity of the site of the focus is the second highest level; when X₂ = 3.05-3.80, the severity of the site of the focus is the highest level.

15. A computer-readable storage medium storing a computer program, wherein the computer program implements steps of the rating method of claim 10 when executed by a processor;
preferably, the computer-readable storage medium is a computer hard disk or a USB disk.
